(19) United States
(12) Patent Application Publication
(10) Pub. No.: US 2006/0052689 A1
(43) Pub. Date: Mar. 9, 2006

(54) Digital stereotaxic manipulator with interfaces for use with computerized brain atlases

(76) Inventors: Charles W. Scouten, Downers Grove, IL (US); James G. Unnerstall, O'Fallon, MO (US); John M. Thompson, Troy, IL (US)

Correspondence Address: Patrick D. Kelly, 11939 Manchester #403, St. Louis, MO, 63131, US

(21) Appl. No.: 10/918,544

(22) Filed: Aug. 14, 2004

(51) Int. Cl. A61B 5/05

(52) U.S. Cl. 600/417

(57) ABSTRACT

A system is disclosed that allows stereotaxic procedures being performed on lab animals to interact, on a “live” or “real-time” basis, with information that has been compiled in stereotaxic brain atlases. For example, during an invasive procedure, a researcher can see, on a cross-sectional brain map displayed on a full-sized computer monitor, the location and travel of an instrument tip, indicated by means such as a bright blinking cursor or icon. If desired, important brain structures (such as major nerve bundles) can been prominently labeled and/or colored, to clearly indicate their locations, and help the researcher ensure that they are avoided. This system can be provided by coupling a digital stereotaxic manipulator to a dedicated controller with touch-screen capability, and coupling the PLC processor to a computer having a monitor screen that is large enough to display a brain map with good resolution. Alternately, the digital stereotaxic manipulator can be coupled directly to a computer, via an interface card or other device.

### FIELD OF THE INVENTION

This invention relates to equipment used in biological and medical research that uses small animals, such as rats or mice. In particular, it relates to electromechanical devices, called stereotaxic holders, that can be coupled to computers.

### BACKGROUND OF THE INVENTION

Background information on stereotaxic holders used in neurological research on rats, mice, and other small animals is provided in parent application Ser. No. 10/636,899, cited above, invented by the same inventors herein, and assigned to Coretech Holdings, which also owns all rights to this current application. The contents and teachings of that parent application are incorporated herein by reference, as though fully set forth herein.

In order to establish component names and callout numbers used herein, FIG. 1 herein depicts a conventional prior art non-digital stereotaxic holder and manipulator, of the type that was used with essentially no changes for nearly 40 years, from about the mid-1950's to the mid-1990's. The main components or subassemblies of the holder include base plate 102, U-frame 104, ear bars or pins 110 and 112, and snouth clamp 121. A manipulator base 184 is securely affixed to U-frame 104. Base 184 supports manipulator system 200 (shown in more detail in FIG. 2), which comprises horizontal slide 190, vertical arm 240, travelling block 260, and horizontal arm 270. An instrument 300 is affixed to the end of horizontal arm 270, thereby allowing the instrument to be moved, in all three linear orthogonal directions, by rotating control knobs 182, 241, and 271, shown in FIG. 2.

The three orthogonal directions that are established by this mechanism are indicated by the arrows in the lower left corner of FIG. 1. These are the anterior-posterior (A/P) axis (horizontal, nose-to-tail), the medial-lateral (MIL) axis (also horizontal, left-right), and the dorsal/ventral (D/V) axis, which is vertical. Using conventional algebraic notations, these axes are sometimes referred to as the horizontal X and Y axes and the vertical Z axis, as shown in FIG. 1.

It should be mentioned, in passing, that the horizontal slide is regarded herein (and by most researchers) as a part of the manipulator. However, because most manipulators are designed and built to allow most of the manipulator to be lifted off the horizontal slide by unclamping turret base 202, some people occasionally refer to a “manipulator” as being limited to the components above the turret base 202, which can be easily and conveniently unclamped and lifted off from what they regard as the base components. The question of whether horizontal slide 180 is part of the base assembly 100, or part of the manipulator assembly 200, is unimportant, and is merely a semantic distinction to anyone who understands how these systems operate.

Until the mid-1990's, stereotaxic manipulators in animal research used relatively crude mechanical vernier readout scales. They had to be inspected visually to obtain useful data, and they were accurate only to a level of 0.1 millimeters, which is 100 microns (roughly 10 times the diameter of a typical mammalian cell, and roughly 100 times the diameter of a neuronal axon or other fiber). Obtaining accurate and reliable readings, for all three vernier scales on all three orthogonal axes, is tedious, time-consuming, and often awkward and difficult, especially in tests carried out in cluttered and crowded settings, or under a hood or other enclosure or equipment. The reading of “Vernier scales” (which is necessary to provide an accuracy level down to 0.1 mm) also can be confusing. In addition, the types of calculations (mainly subtraction) that are required to determine positions relative to an arbitrary “zero spot” called the bregma (located at the intersection of two “fissures” or “sutures” that are visible on the top of an exposed rat skull) are tedious and prone to error, when vernier scales are used and numerous digits must be manually punched into some type of keypad.

It was a major advance when digital electronic readers, originally developed for digital calipers and other measuring instruments, were adapted for use with stereotaxic manipulators. This was first accomplished by a company called Cartesian Research, in the mid-1990's. In addition to providing digital readout capability that could be accurate down to a single micron (roughly 100 times more precise than can be provided by vernier scales that must be inspected visually), the digital manipulators created by Cartesian Research also provided automated “zero-ing” capability. This allowed location values in all three orthogonal axes to be set to zero values, when an instrument tip touched the bregma location, on the top of an animal skull, at the start of a procedure. After the digital display device was reset to “zero” values when the instrument tip was at the bregma location, all subsequent values were then displayed as values that directly indicated orthogonal distances from the bregma location, without requiring any additional calculations.

It should be noted that the term “procedure” is used broadly herein, to include any type of intervention, emplacement, data-gathering, or other procedure which is performed on a non-human animal with the involvement of a stereotaxic manipulator.

Any reference herein to animals is limited to non-human animals, and stereotaxic manipulators as used herein are limited to systems used in research, rather than surgery in human medicine. It should be recognized that extraordinarily complex, expensive, and sophisticated systems have been developed for certain types of surgery on humans, especially on human brains, spinal cords, and hearts. However, those are vastly too expensive to justify their use in animal research, which faces entirely different economic and competitive limitations and boundaries. Among other factors, the market for stereotaxic manipulators is small and constrained, largely due to two facts: (i) there is only a limited number of laboratories that perform invasive research into small animal brains, and (ii) once a laboratory that performs that type of research has purchased a holder and manipulator system, that system is highly durable, does not wear out, and can be used in many thousands of tests over a span of decades.

In addition, complexity and training impose two other major constraints on how complex or sophisticated a stereotaxic manipulator can be, for use in research on small animals. The training that is required, before a surgeon can use a complex computerized machine in human surgery, utterly dwarfs the training that can reasonably be expected, or provided, before a researcher begins trying tests on mice or rats.

For both sets of reasons, surgical devices developed for human surgery are not deemed to be relevant, as prior art, in considering the invention herein.

The digitized systems that were developed by Cartesian Research were the first commercially-available digital stereotaxic manipulators. The term “stereotaxic manipulator” (also referred to simply as a manipulator, for convenience) is used herein in its conventional sense, as understood by researchers who use non-human animals in their tests. The adjective “digital” (or similar terms, such as digitized, etc.) indicates that a manipulator has one or more electronic components that can emit electronic signals indicating the motion (or travel, displacement, or similar terms) and/or position (or location, coordinates, etc.) of an instrument that has been mounted on the manipulator arm, along three orthogonal axes. In order to be useful and practical, a digital manipulator must be able to emit those digital signals on a “live” or “real-time” basis, during the course of a procedure, to give the researcher a clear indication of where an instrument tip is actually located, at any particular moment in time.

The first digital stereotaxic manipulators offered an important and highly useful advance. However, the earliest models suffered from several limitations. They were relatively large and cumbersome, which are major disadvantages in most laboratory settings, which almost always involve crowded benchtops. They also were relatively expensive, and a new complete system (including the baseplate, manipulator mount and slide, etc.) had to be purchased, with no means for retrofitting already-owned manipulators. For those and other reasons, sales of the earliest models were limited, and the Cartesian Research company was later purchased by another maker of stereotaxic manipulators.

In 2001, Coretech Holdings and one of its subsidiaries, myNeuroLab, developed an improved stereotaxic manipulator, having digital scales and readers that were small enough to be retrofitted, conveniently and inexpensively, to the existing base plate and holder of most types of conventional stereotaxic manipulators, without increasing the “footprint” size of the system or the amount of space it had to occupy on a benchtop. That advance is described and illustrated in US utility application Ser. No. 10/036,231, filed on Dec. 24, 2001 by Scouten et al (published on the U.S. PTO website under accession number 2003/0120282). The contents and teachings of that application also are incorporated herein by reference, as though fully set forth herein.

That type of digital manipulator system is illustrated in FIG. 2, which is identical to FIG. 2 in application Ser. No. 10/636,899. This drawing illustrates electronic reader heads 514, 554, and 574, which are affixed adjacent to linear etched scales 502, 542, and 562, respectively. Whenever the slide 180, vertical arm 240, or horizontal arm 270 is operated by one of the control knobs 182, 241, or 271, respectively, relative motion will be generated between a reader head and its accompanying linear scale. Reader heads 512 (on slide 180) or 574 (on horizontal arm 270) will remain stationary while linear scales 514 and 562 (respectively) move beneath them, when the slide or horizontal arm are moved. By contrast, reader head 554 will travel vertically when the “travelling block” 260 is moved vertically, while vertical linear scale 542 remains stationary.

During operation, the relative motion between a linear scale and a reader head enables the reader head to generate and emit a digital electronic signal, which precisely indicates the amount of relative travel between the two components. This digital signal can be sent to a small processor unit, or a full-power computer, which can process those digital signals to determine the extent and direction of the travel, to an accuracy of 1 micron or less. These digital measurements can be displayed on the display or monitor screen of the processor or computer.

As used herein, the term “computer” as used herein includes any processing system that is designed to facilitate operations in which an owner or operator can load any type of desired software into a memory and/or storage device, such as a hard drive. By contrast, the term “processor” is used herein to refer to devices that are preloaded by a manufacturer or seller with only certain limited types of software, and that are not designed to enable simple loading or use of other types of software by an owner or operator.

After application Ser. No. 10/036,231 was filed in December 2001, Coretech Holdings and myNeuroLab continued to design and develop digitized stereotaxic manipulators with more improvements. Five important advances they created are described in above-cited application Ser. No. 10/636,899, filed in August 2003. Those enhancements, illustrated in FIGS. 3 and 4 herein (which are identical to FIGS. 3 and 7 of application Ser. No. 10/636,899) are not considered to be prior art against the invention disclosed herein, and instead should be regarded as part of this current invention, since this invention builds upon those prior disclosures and describes improved methods for making better and more effective use of those enhancements.

One of those enhancements was a “fine drive” mechanism, illustrated in a perspective view by components 3700, 3712, and 3730 of FIG. 3. This subassembly uses a cylindrical “worm gear” to drive and control a radial “star gear” that is affixed to the main vertical shaft 242 (shown in FIG. 2) of the enhanced manipulator 3000. This fine-drive mechanism 3700 allows much more accurate control (with roughly 20 times greater precision) over vertical motion of the instrument than could be obtained previously. It is illustrated and explained in detail in other drawings in application Ser. No. 10/636,899. Since it is not essential to other enhancements disclosed herein, it is not addressed in detail herein.

A second major enhancement involves at least one and preferably two “rotary encoders”, to provide precisely-measured angular control over at least one and preferably two of the arms of a manipulator. As illustrated in FIG. 3, rotary encoder 3102 is mounted beneath vertical arm 240 of manipulator 3000. Encoder 3102 allows precise measurement of the rotation, about a vertical shaft, of turret base 202 and vertical arm 240 (as well as the horizontal arm 270 and the travelling block 260, which are supported by the vertical arm 240). Using a relatively inexpensive mass-produced device for rotary encoder 3102, the angular displacement (or partial rotation) of the vertical arm assembly 240 can be measured to an accuracy of about ⅙ of an angular degree. This level of accuracy is entirely adequate for most purposes; if desired, even greater accuracy can be provided, by more expensive encoders, or by other mechanisms (for example, an interacting worm-gear and radial-gear arrangement can be used to provide a similar “fine-drive” mechanism, as briefly described above and in more detail in application Ser. No. 10/636,899).

In FIG. 3, the horizontal arm 270 is shown in an orientation that is parallel to horizontal slide 180. That is done for illustration purposes only; during use of the manipulator 3000, the horizontal arm 270 will be perpendicular to the slide 180, as shown in FIGS. 1 and 2.

In the preferred embodiment shown in FIG. 3, a second rotary encoder 3202 is also used to establish a horizontal axis, as part of the so-called “turret base” subassembly that supports the vertical arm 240. This allows the vertical arm 240 to be tilted away from true vertical, to any desired slanted angle, while the angle of tilt is accurately measured by rotary encoder 3202.

By combining controlled and accurately-measured rotation of both of the two rotary encoders 3102 and 3202, a wide variety of slanted (or angled, tilted, etc.) options and approach paths become available, for an instrument tip that is being maneuvered and controlled during a procedure, by manipulator 3000.

The third major enhancement was a decision and commitment to create a digital processor and display device (exemplified by item 3900, illustrated in FIG. 4 herein, which is identical to FIG. 7 in application Ser. No. 10/636,899) with enough internal computing power and memory, and with sufficient embedded software to handle various types of trigonometric and other mathematical algorithms and calculations. Those calculations are performed on data that is being supplied by five different digital readers, which include the three linear electronic reader heads 514, 554, and 574 (shown in FIG. 2) and the two rotary encoders 3102 and 3202 (shown in FIG. 3).

By using sine and cosine values (which will depend on the rotated or tilted angles of the two rotary encoders 3102 and 3202, at any moment during a procedure), the “apparent” orthogonal values that are being measured and emitted by the three linear reader heads 514, 554, and 574 can be converted, by the software algorithms inside the dedicated processor 3900, into “angle-adjusted” values that are accurate and reliable, in all three true orthogonal axes, on a “live” or “real-time” basis, at any moment during a procedure. Accordingly, “angle-adjusted” (corrected) values for all three true orthogonal axes can be displayed, live and in real time, during a procedure, even when the manipulator has been rotated, angled, and tilted in ways that cause it to deviate from the true orthogonal axes.

The fourth major enhancement was a decision and commitment to provide the digital processor and display device 3900 with a display panel (indicated by callout number 3920 in FIG. 4) that provides “touch-screen” capability. This allows “virtual buttons” to be displayed on the touch-screen panel 3920. When a “virtual button” is touched by an operator, it will activate a sensor that will trigger any command or control action that has been programmed into the software that has been loaded into the processor. These types of “touch-screen” displays are used on many types of programmable cash registers, on most “personal digital assistant” (PDA) devices, and on various other electronic devices. Among other advantages, they can eliminate the need for a separate keypad or other device with keys, for inputting commands. By calling up a main menu that provides various options (including the option to call up submenus, which can be unlimited in their number and variety), the use of touch-screen capability, in a small dedicated digital processor, can enable a small dedicated processor to be used for controlling a greater range and assortment of tasks than could otherwise be provided by a compact and relatively inexpensive unit.

The fifth major enhancement was a decision and commitment to provide the dedicated processor and display device 3900 with a data output port (illustrated by output port 3916 in FIG. 3) which will allow the processor to be connected directly to a “full-power computer”. The term “full-power computer” is used herein to include any type of conventional computer that can be loaded with any of numerous different types of software (by comparison, dedicated processors can be loaded with only one or a limited number of software programs). Full-power computers can be, for example, conventional desktop, laptop, or notebook computers, which typically will having a hard drive, a fast processor chip, and a color monitor or display screen, as well as various means for loading software and transferring data into the computer. The data output port 3916, on the dedicated processor and display device 3900, can be, for example, a conventional “universal serial bus” (USB) port, which has become a preferred and widely used input/output system for computer peripherals. Alternately, other types of ports (such as a serial, parallel, or “Firewire”0 port) can be used, but those generally are less adaptable than USB ports.

The ability to transfer data from manipulator 3000 to a full-power computer (either through a dedicated processor 3900 having a data output port 3916, or by sending electronic signals from a manipulator directly to a computer via a dedicated “card” or other adapter or device), can allow a computer to process and display data from the manipulator, in real time, during the actual course of a procedure, in ways that cannot be provided by a small processor and display device having a small low-cost display screen.

This combination, as described in above-cited parent application Ser. No. 10/636,899, enables controlled, precise, and digitally-measured rotation of a manipulator arm and instrument about both a vertical axis, and a horizontal axis, using rotary encoders that emit digital signals to indicate their precise angular displacements at any time during a procedure. It also enables a small dedicated processor with touch-screen capability to provide accurate (angle-adjusted) orthogonal locations of an instrument tip, in real time, throughout the course of a procedure.

Those combined enhancements provided highly important, useful, and quickly-recognized benefits, allowing options and approaches that were not available with any previous stereotaxic manipulators. An example of how and why that enhanced system provided important advantages over prior manipulators can be seen from the following illustration, which addresses a fairly common problem. Because mammalian brains are highly symmetric with respect to their right and left hemispheres, there are numerous highly important brain regions that sit directly between the hemispheres, rather than being part of one side or the other. These brain structures sit directly upon, and straddle, the vertical “center sagittal plane” that divides the brain into left and right hemispheres. Examples of such center-plane structures that are highly important in neurological research include the hippocampus, the medial thalamus, the suprachiasmatic nucleus, and the raphe nuclei.

However, there is also a large blood vein, called the superior sagittal sinus, positioned directly on top of the center of the brain, running front-to-back along the center sagittal plane. This large vein rests in the large crease that naturally exists between the upper surfaces of the left and right hemispheres of the brain in nearly all mammals, ranging from rodents to humans. If that major vein is punctured during an invasive procedure, it will release large quantities of blood, most likely killing the animal, or inflicting major brain damage on it.

Therefore, if a researcher wishes to insert an instrument tip or needle in or near the center sagittal plane, to study one of the brain structures that sits between the two hemispheres and serves both hemispheres, steps must be taken to somehow angle and offset the approach path of that instrument or needle, to avoid puncturing the superior sagittal sinus blood vein. However, if steps are taken to avoid that vein by using an angled approach, the angled approach will distort any orthogonal readings that are taken by any type of manipulator that does not allow precise angular measurements and calculations.

To develop their enhanced system to a “first plateau” level, in which the dedicated processor and display device 3900 would be fully functional and useful even though it would not yet be coupled in a truly effective manner to a computer, Coretech Holdings and myNeuroLab hired a contractor company that specializes in writing software for “programmable logic controller” (PLC) devices. That contractor company wrote source-code software that was loaded into programmable memory chips, using a “flash memory” method that does not allow the source code to be modified unless special steps are taken. The chips were then inserted into the processor and display units, and the units were ready for sale, to accompany enhanced manipulator systems that were equipped not just with linear digital readers, but also with rotary encoders as well.

As soon as this system was announced, publicly displayed, and offered for sale at a major trade show, under the trademark “Angle One,” the combination of hardware and software created a realization among potential users (and among competing manufacturers of less-capable stereotaxic equipment) that it offered a major advance over any other stereotaxic holders that were previously available for use with small animals. Within just a few months, the new “Angle One” system rapidly established a position as the new standard, for improved stereotaxic manipulators.

This completes a brief summary of the advances disclosed in U.S. application Ser. No. 10/036,231 (which describes small and convenient but precise linear digital readers that can be retrofitted to conventional stereotaxic holders), and application Ser. No. 10/636,899 (which describes rotary encoders and programmable display devices that can calculate and display accurate orthogonal values, even when a manipulator has been rotated or tilted about vertical and/or horizontal axes).

This current application discloses another important advance and enhancement, which complements and extends the digital capabilities disclosed in U.S. application Ser. Nos. 10/036,231 and 10/636,899.

Now that the inventors herein have managed to reach an important and useful plateau in this field of technology, it has become feasible to provide another important enhancement for their manipulators and processors, to enable them to interact directly with computerized media containing information that has already been compiled in “brain atlases”.

This new development, which forms the basis of this invention, requires an understanding of how brain atlases are organized, formatted, and used. Accordingly, that background information is provided below.

Brain Atlases

Brain atlases have been compiled for all of the animal species that are widely and commonly used in neurological research. Because of differences between species, a separate atlas is required for each different species of animal used in such research. Therefore, brain atlases have been created and published for mice (e.g, Paxinos & Franklin 1999), rats (e.g., Paxinos & Watson, 1998), golden hamsters (e.g., Morin and Wood 2001), rhesus monkeys (e.g., Paxinos, Huang, & Toga 1999) and various other species. These atlases are available both in printed form (on paper), and as computerized media on read-only compact discs (CD-ROM's). The printed and computerized versions are usually published and sold together, with the CD-ROM tucked into a pocket in the book that contains the printed versions of the same pictures.

The printed book versions usually are bound by spiral or other plastic holder rings, rather than glued book spines, to enable the owner to photocopy pages (for convenient use during a procedure) without bending, cracking, or damaging a glued spine. The versions on CD-ROM's contain picture files, each containing a single high-resolution picture that can be displayed on a computer monitor, using software that provides zoom-in capability for closer inspection of enlarged portions of a picture.

Printed and/or computerized brain atlases follow a consistent format and organization. Briefly, these involve pictures of brain tissue cross-sections, taken at a succession of closely-spaced intervals or increments, along the anterior-posterior (A/P, front-to-rear, nose-to-tail) axis of the animal brain. Each picture contains and displays a single vertical cross-section, corresponding to one particular location along the A/P axis. In each picture, the top of the brain is shown near the top of the picture, and the left side of the brain is shown on the left side of the picture. Because of their two-dimensional layouts, any such cross-sectional drawing can also be referred to as a “brain map”.

Three examples of rat brain maps are provided in FIGS. 5, 6, and 7. These pictures are prior art, and are from The Rat Brain in Stereotaxic Coordinates, fourth edition with compact disc, by George Paxinos and Charles Watson (Academic Press, 1998). FIG. 5 shows a forebrain section, at a vertical “coronal” plane located 4.70 mm anterior to the bregma plane (the bregma plane corresponds to the “0” location indicated in the smaller drawing in the upper left corner of FIG. 5). FIG. 6 shows a midbrain section, located 4.80 mm posterior to the bregma plane. FIG. 7 show a hindbrain section, located 12.30 mm posterior to the bregma plane.

Similar drawings are available in brain atlases for mice, hamsters, sheep, and other species. Brain atlases for primates and certain other animals have somewhat different appearances, due to their rounder brain structures, but they are organized and formatted in the same manner described herein.

Three of the main structures of rat brains, which are shown in the small sagittal cross-sectional drawings in the upper left corners of FIGS. 5-7, should briefly be mentioned, since they can help explain the utility of this invention in certain types of research. The bulb-shaped component at the very front of the brain is the olfactory bulb, and it is heavily involved in the sense of smell. Since an acute sense of smell is essential for helping rats finding food, their olfactory bulbs are roughly the same size as the olfactory bulbs in humans.

The largest upper segment, which extends from roughly 5 mm anterior to the bregma plane, to about 8 mm posterior to the bregma plane, is the cerebral neocortex. A relatively large upper cleft, located about 8 to 9 mm posterior to bregma, separates the cerebral neocortex from the cerebellar cortex, which extends from about 9 to about 15 mm posterior to bregma.

The large upper cleft is worth noting, since certain types of angled approaches that are enabled by this current invention (but which would be extremely difficult to carry out accurately, using prior art manipulators) may allow an instrument tip to enter the brain through that cleft, in a way that can avoid and spare both the cerebral neocortex, and the cerebellar cortex.

The pictures in FIGS. 5-7 are rendered in black-and-white line drawings, which were created mainly by ink tracings that were drawn over enlarged photographs. Each of the line drawings in FIGS. 5-7 is labeled with numerous acronyms, which refer to various regions of the brain. Most acronyms are superimposed on either the left or right hemisphere, but not on both; since the brain is highly symmetric about the center vertical (sagittal) plane, there is no need to place the same acronym on both sides, and doing so would clutter up the drawings and render them more difficult to interpret.

While any skilled neurology researcher will memorize and quickly recognize the names and acronyms for several dozen important brain regions, there are hundreds of named structures in rodent brains, and almost no one bothers to memorize all of those structures and their acronyms, since many of them are rarely of interest in any research. Therefore, some printed atlases contain, on each line-drawing page, a listing of all acronyms and structures shown in that particular drawing, giving the full name for each acronym. More commonly, a complete alphabetized index usually is provided in the back of each atlas, which compiles all of the acronyms used in the atlas, along with their complete names, and a listing of all pages on which each structure appears.

In FIGS. 5-7, the solid dark regions are ventricles, which are filled with cerebrospinal fluid (CSF) rather than tissue. CSF is generated near the front of the brain, and slowly flows through the brain in a posterior direction, toward and into the spinal cord.

A number of distinct “nerve fiber bundles” pass through the brain, which carry nerve signals between the brain and various muscles, internal organs, etc. One example is the “mammillothalamic tract” (mt) bundle, labeled near the bottom of FIG. 6, to the left of the centerline (the sagittal plane; nerve bundles generally are labeled with small-letter acronyms, in most atlases). Many of these nerve bundles need to be avoided, during most types of invasive procedures, since puncturing or lacerating an important bundle can kill, paralyze, or otherwise severely injure an animal, and may render worthless any effort to gather useful data from that animal.

As can be seen in FIGS. 5-7, each cross-sectional drawing is positioned in a square grid, which is numbered along the vertical and horizontal axes. The numbers along the vertical axis on the right sides of the drawings represent vertical distances (dorsal-ventral, D/V) from the height of the horizontal bregma plane. Since the bregma is located on the top of the skull, those vertical numbers increase as the distance from the top of the grid increases.

Different numbers are provided along the left axes, and those numbers increase as they get higher. These numbers usually indicate vertical distances relative to the horizontal plane of the ear bars or pins, when an animal is in a stereotaxic holder. There is greater variability (and less precision) in “ear bar zero” numbers than in bregma numbers, so “ear bar zero” numbers are used infrequently in most research on the brain. Nevertheless, they can be relevant in certain types of experiments, and it is convenient to have them shown and available, if needed.

The numbers along the horizontal axes are “zero'ed” at the midpoint, which corresponds to the center sagittal plane, which passes vertically through the center of the brain and separates the brain into two hemispheres. The horizonal grid numbers grow larger, as the distance from the sagittal plane increases in either the left or right direction.

Each digit, along the vertical or horizontal axes of the grids, corresponds to exactly one millimeter, in distance, in the brain. Since most printed atlases for mice, rats, or hamsters use grid spacings of 1 centimeter, those drawings are enlarged by a factor of 10:1 (linear). Atlases for larger animals (such as pigs or monkeys) are often printed on oversized paper, and different sizing factors may be used.

Most brain atlases that are printed on paper also provide, on facing pages that can be seen whenever the atlas is opened to a certain particular cross-section, a photographic image that corresponds to that cross-sectional line drawing at that particular location along the A/P axis of the brain. This photographic image will show enlarged photographs of the left and the right sides of a normal and healthy brain, placed adjacent to each other, as would be seen in an intact brain. However, to provide even more potentially useful information, the two halves (or alternate sections) of these photographs (which are almost always perfectly symmetric) depict the results of tissue staining, using two different types of stains. Most commonly, the left half of the stained section photograph shows the results of fiber staining, or a specific neurochemical stain, such as an “AChe” (acetylcholinesterase) stain, while the right half of the stained section photograph shows the results of Nissl (cell body) staining, using a dye called Creysl Violet.

This completes an overview of how brain atlases are organized, formatted, and presented.

As mentioned above, computerized atlases for nearly all types of lab animals used in neurological research are available, on compact discs. However, prior to this invention, the technology has not been available for providing useful and helpful operations, during a stereotaxic procedure on an animal, that can make good use of computerized information available in brain atlases.

Accordingly, one object of this invention is to provide methods for utilizing information contained in a computerized brain atlas, in conjunction with a stereotaxic manipulator having a computerized interface, to help guide, control, and improve a stereotaxic procedure that is being performed on a lab animal.

Another object of this invention is to utilize computerized brain atlas information, in conjunction with a stereotaxic manipulator having a computerized interface, to provide various types of information that will be useful, on a “live” or “real-time” basis, during a stereotaxic procedure on a lab animal.

Another object of this invention is to utilize computerized brain atlas information, on a “live” or “real-time” basis in conjunction with a stereotaxic manipulator having a computerized interface, to provide various types of information that will be useful to help a researcher guide and maneuver an instrument tip to an exact targeted location, by using a predetermined route that will inflict the least possible amount of damage on the animal's brain and nervous system.

Another object of this invention is to utilize computerized brain atlas information, on a “live” or “real-time” basis in conjunction with a stereotaxic manipulator having a computerized interface, to inform a researcher, at any time during a stereotaxic procedure, exactly how far away an instrument tip is from any particular point in the brain that has been selected and input by the researcher, along all three orthogonal axes.

Another object of this invention is to utilize computerized brain atlas information to allow a researcher to operate a “display the nearest cross-sectional picture” control, at any time during a procedure, which will then cause a high-resolution line drawing or photograph to appear on a computer monitor screen, corresponding to the current position of the instrument tip along the A-P axis of the brain, and showing a blinking, colored, bright pinpoint, or other cursor-type indicator light or icon that will be superimposed on the drawing or photograph, to indicate the location of the instrument tip at that moment in time.

Another object of this invention is to provide computerized systems that can display, during a stereotaxic procedure, enhanced versions of computerized brain atlas drawings, having features such as (i) color-coded nerve bundles or brain structures, and/or (ii) nerve bundles or brain structures that will emit special signals (such as blinking colors, audible alarms, etc.) if they are approached too closely by an instrument tip, if they are directly in the path of an instrument, of if they otherwise are being jeopardized during a procedure.

Another object of this invention is to provide a computer interface with a stereotaxic manipulator, which can calculate an exact bregma location and then allow the operator, to place an instrument tip at that exact location, rather than relying on a visual “best guess” to determine an estimated bregma location that may be misplaced by hundreds of microns.

Another object of this invention is to provide a stereotaxic manipulator with a rotatable snout clamp and a rotary encoder, positioned to allow precise measurement of the rotation of an animal about an anterior-posterior axis, thereby allowing an animal to be tilted, or rotated onto either side or its back, in ways that can facilitate various types of procedures while still providing precise orthogonal measurements that have been adjusted to accommodate for the angle of rotation of the animal.

These and other objects of the invention will become more apparent through the following summary, drawings, and description.

### SUMMARY OF THE INVENTION

Computerized hardware and software can allow stereotaxic procedures being performed on lab animals to interact, on a “live” or “real-time” basis, with information that has been compiled in stereotaxic brain atlases, which have been prepared for various species of lab animals used in neurology research. As one example, during an invasive procedure, a researcher can see, on a cross-sectional brain map displayed on a full-sized computer monitor, the location and travel of an instrument tip, indicated by means such as a bright blinking cursor or icon. If desired, important brain structures (such as major nerve bundles) can been prominently labeled and/or colored, to clearly indicate their locations, and help the researcher ensure that they are avoided. This type of interactive display of information, shown on a “live” or “real time” basis on a computer monitor, can help a researcher guide an instrument tip to an exact targeted location, via a predetermined route that will inflict the least possible damage on the animal's brain. The computer can be programmed to display continuous updates on the distance from the instrument tip to the targeted location, both in terms of distances (measured in microns) and by displaying bright, blinking, or similar cursors or icons for both the instrument tip and the targeted site. If desired, enhanced computerized atlases can be provided that will cause nerve bundles or brain structures displayed on a monitor to blink, or trigger an alarm signal, if they are jeopardized by an instrument during a procedure.

This enhanced system can be provided by (i) coupling a digital stereotaxic manipulator to a dedicated “programmable logic controller” (PLC) processor with touch-screen capability, which can convert data from linear readers and rotary encoders into angle-adjusted data showing orthogonal locations in real time during a procedure; and,. (ii) coupling the PLC processor to a desktop, laptop, or other computer having a monitor screen that is large enough to display a brain map with good resolution. Alternately, the digital stereotaxic manipulator can be coupled directly to a computer, via an interface card or other device.

### BRIEF DESCRIPTION OF THE DRAWINGS

FIG. 1, which is prior art, discloses a conventional animal holder, with a stereotaxic manipulator that uses vernier scales that must be inspected visually), for use with small animals such as rats.

FIG. 2 illustrates a digital stereotaxic manipulator, as described in copending application Ser. No. 10/036,231, which belongs to the same assignee company herein. That system contains three electronic reader heads that are mounted next to adjacent linear scales, so that operation of any of the slide or arms of the manipulator will cause the reader heads to send signals to a separate display unit that will (i) display digitized position data for each of the three orthogonal axes, and (ii) provide convenient “zero-ing” capability, so that all coordinates can be set easily to zero values when an instrument tip is at a “baseline” point, such as the bregma location on a rat or mouse skull.

FIG. 3 is a perspective view of an assembled third-generation digitized stereotaxic manipulator, showing two “incremental encoders” (also referred to herein as “rotary encoders”) that can be used to measure, to within about ⅙ of a degree, the position of a component that has been rotated about a vertical or horizontal axis.

FIG. 4 depicts a “programmable logic controller” (PLC) processor and display unit that can display, on a “touch screen” display, measured and calculated positioning data, at any given moment during a stereotaxic procedure on a small animal. The measured values are generated by processing the signals from the three linear reader heads, and the two incremental (rotary) encoders. The calculated orthogonal values are generated by using trigonometric values, which will depend on the angular displacements of the two rotary encoders, to adjust and correct the measured values from the three linear reader heads. This dedicated device is also provided with a data output port, which allows the device to be coupled directly to a full-power computer with a large color monitor screen.

FIG. 5 is a cross-sectional brain map taken from a stereotaxic atlas of a rat brain, showing a forebrain region located 4.70 millimeters anterior to the bregma plane.

FIG. 6 is a cross-sectional brain map taken from a stereotaxic atlas of a rat brain, showing a midbrain region located 4.80 millimeters posterior to the bregma plane.

FIG. 7 is a cross-sectional brain map taken from a stereotaxic atlas of a rat brain, showing a hindbrain region located 12.30 millimeters posterior to the bregma plane.

FIG. 8 depicts a stereotaxic manipulator coupled, via a dedicated processor or other interface device, to a desktop computer that is displaying a rat brain map on the monitor, with a blinking cursor showing the location of an instrument tip on the monitor, at the tip of a “track line” showing the path the instrument tip travelled while reaching that position.

FIG. 9 is a perspective view of a stereotaxic manipulator with a snout clamp that can be rotated about an anterior-posterior axis, provided with a rotary encoder that enables precise measurement of the rotation of the snout clamp.

### DETAILED DESCRIPTION

As summarized above, this invention discloses certain enhancements for a combination device that was previously described in utility patent application Ser. No. 10/636,899, filed in August 2003. That system has been publicly advertised and sold by Coretech Holdings and its subsidiary, myNeuroLab, since shortly after the filing of that application.

Briefly, a “baseline” system that does not include the enhancements of this current invention includes the following major subassemblies and components:

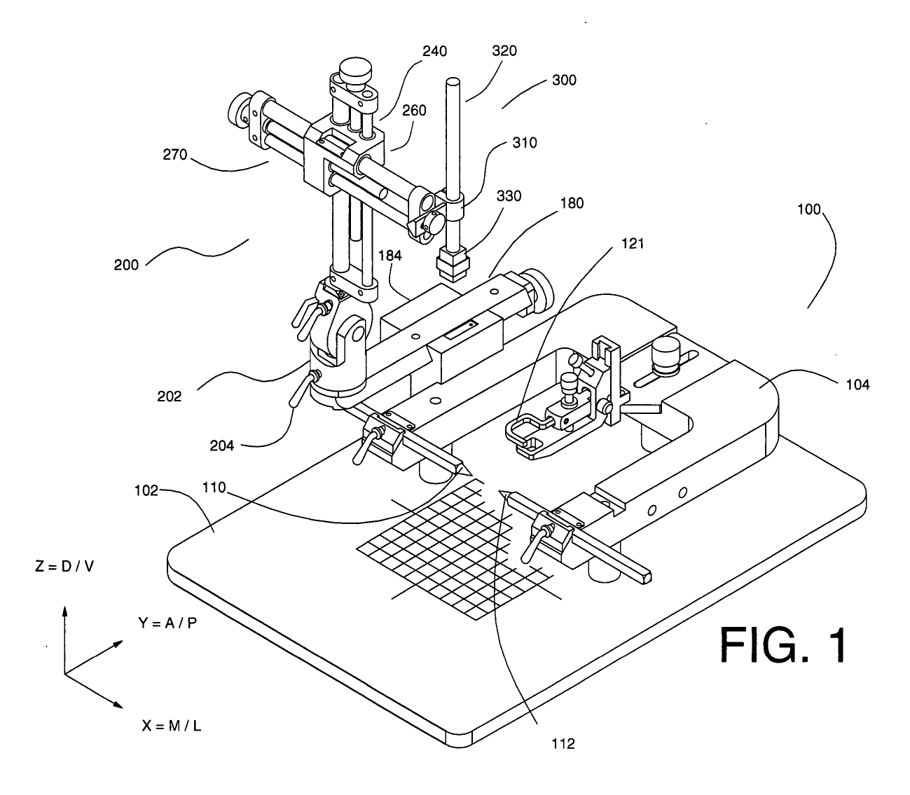

(1) a stereotaxic manipulator 3000 (with a general structure such as shown in FIGS. 1-3) for holding rats, mice, or other non-human animals, which has been equipped with both: (i) linear scales and electronic reader heads that enable digital measurement of linear motion of an instrument tip along all three orthogonal axes, such as scale-and-reader combinations 512 and 514 (on slide 180), 542 and 554 (in vertical arm 240), and 562 and 574 (on horizontal arm 270); and, (ii) rotary encoders that enable precise digital measurement of angular displacement (partial rotation) about a vertical axis and a horizontal axis, such as encoders 3102 (vertical axis) and 3202 (horizontal axis);

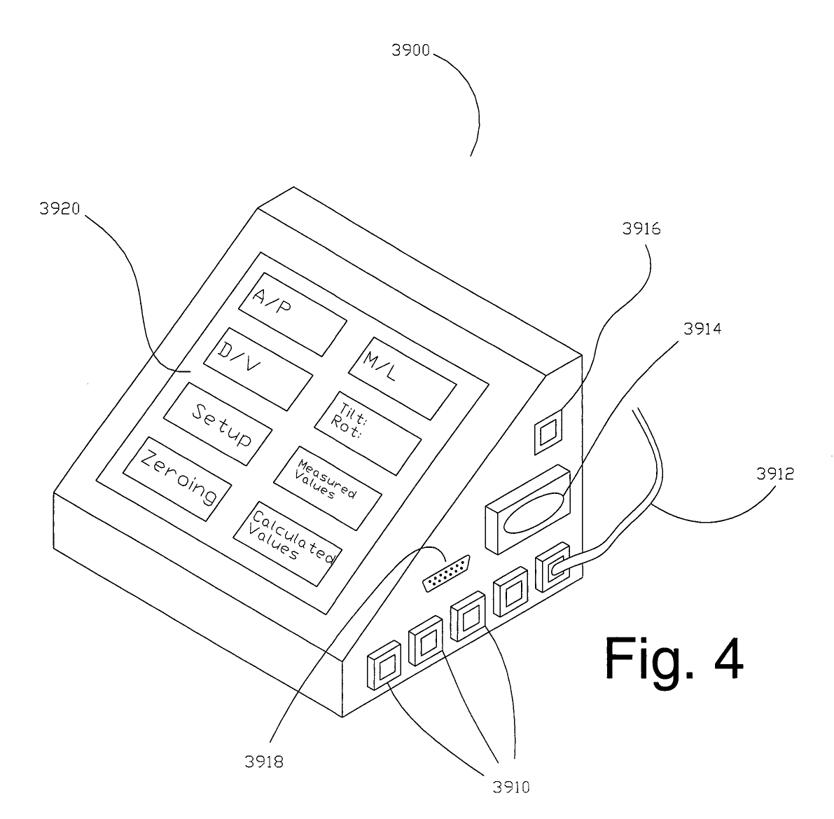

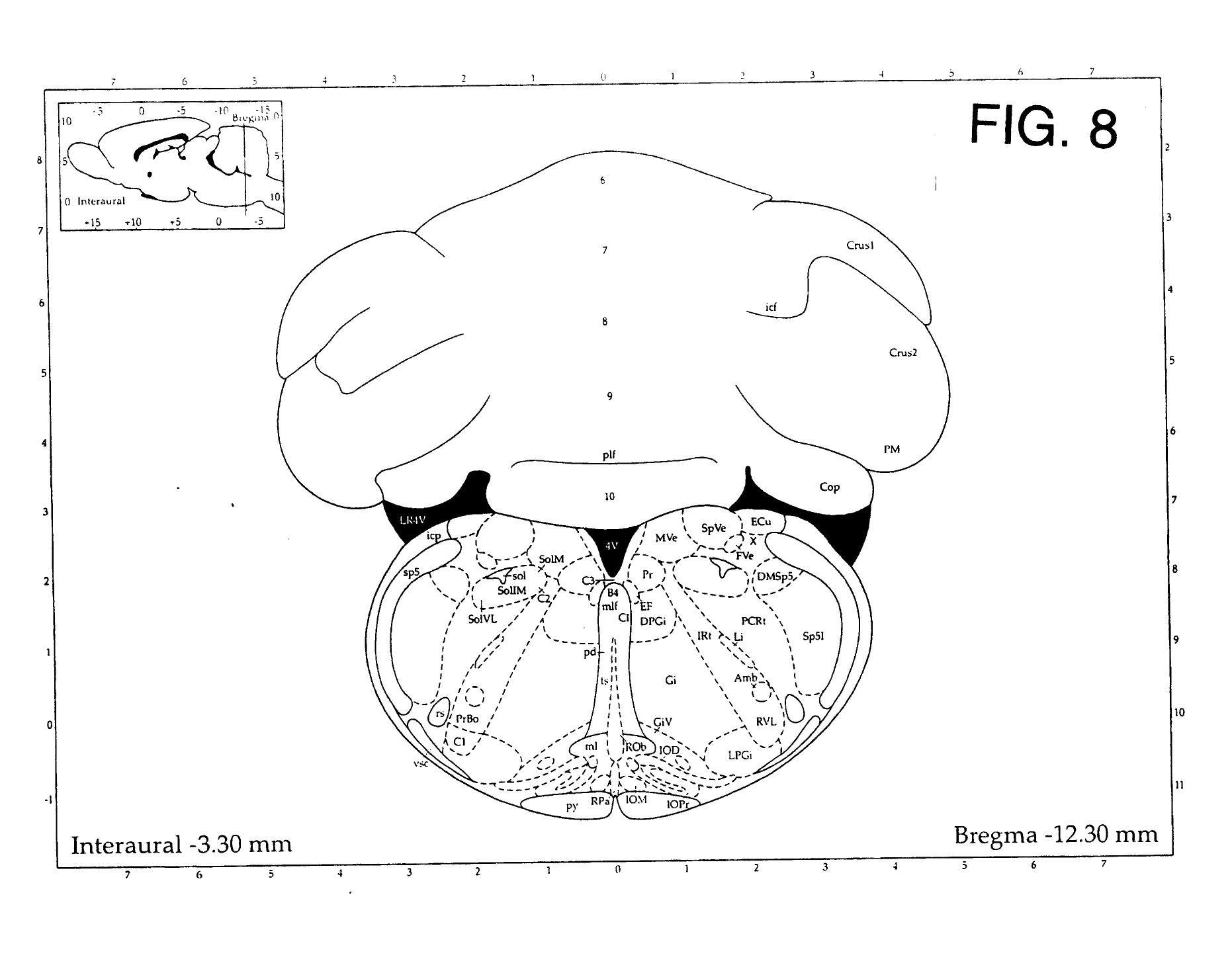

(2) an electronic device that can receive and interpret electronic signals being sent by the manipulator 3000, preferably in the form of either: (i) a dedicated “programmable logic controller” (PLC) device, such as processor 3900 as shown in FIG. 4, having a touch-screen display that can display information and that also can be used to input various commands; or, (ii) a full-power computer, such as desktop computer system 4000 shown in FIG. 8 and described in more detail below; and,

(3) software that has been loaded or embedded into the processor 3900 and/or computer 4000, which enables the processor or computer to display, on a monitor or display screen, apparent (measured) as well as corrected (angle-adjusted) orthogonal coordinates for an instrument tip, relative to a known reference location such as the bregma point on the animal's skull, on a “live” or “real-time” basis (i.e., the information being displayed must be continuously updated, to accurately indicate the current location of an instrument tip at all times during the procedure, even when the instrument tip is being moved).

As mentioned above, that system is described in detail in application 10/636,899, and it is commercially available from myNeuroLab, trademarked as the “Angle One” system. It is regarded herein as a “baseline” system, for the purpose of describing additional enhancements.

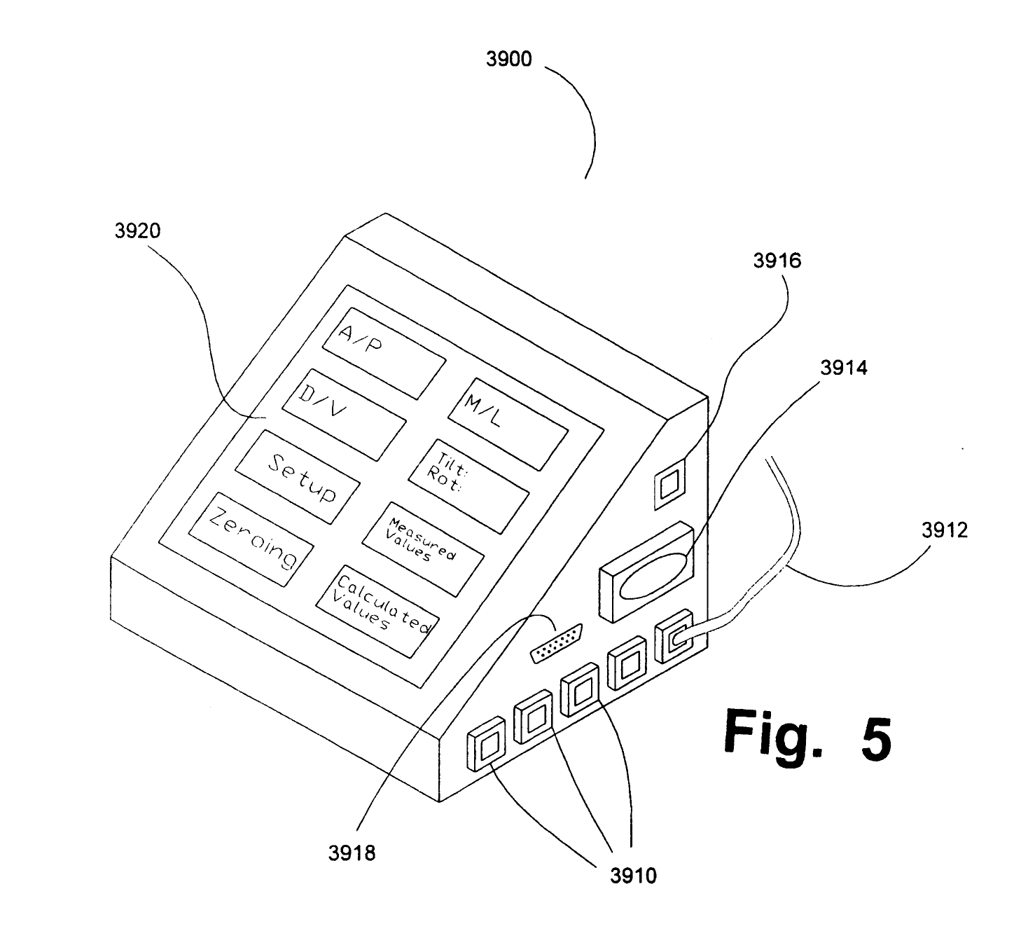

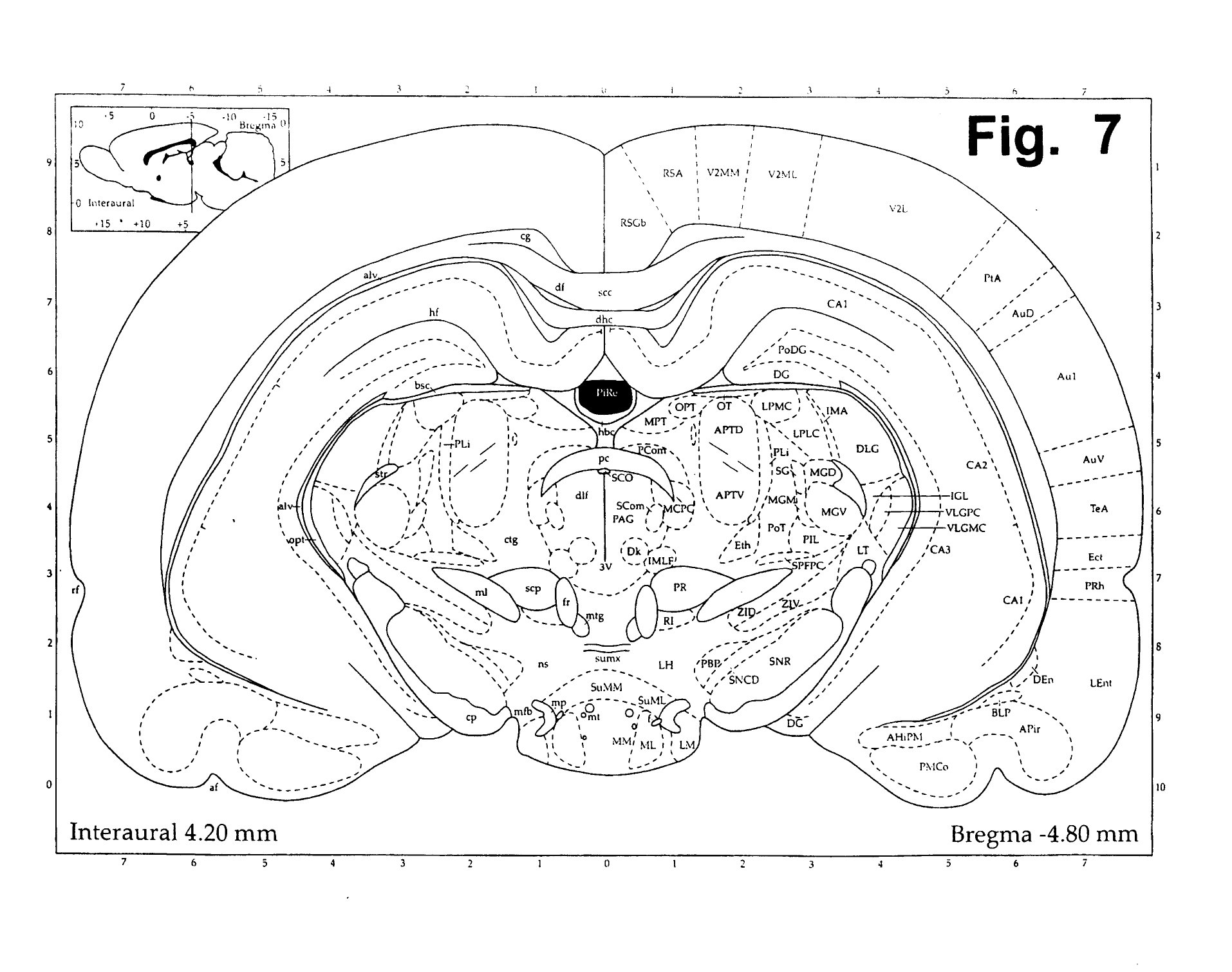

This current invention discloses means for enabling that type of manipulator-processor-software system to interact with maps and pictures that already have been prepared and compiled as “brain atlases”, which comprise a series of cross-sectional line drawings depicting the structures in an animal's brain. As shown by the examples in FIGS. 5-7, described in the Background section and available in publications such as Paxinos & Watson 1998 (rats), Paxinos & Franklin 1999 (mice), and Morin and Wood 2001 (golden hamsters), these types of brain atlases comprise a series of brain maps along vertical coronal planes, spaced apart from each other in small increments along the anterior-posterior (nose to tail) axis. As described in more detail below, a selected brain map that corresponds to the coronal plane that will be involved in an invasive procedure can be displayed on a computer monitor. During the procedure, a blinking cursor, icon, or other representation can be used to indicate the location of the instrument tip, superimposed on the brain atlas, at all times during the procedure, on a “live” or “real time” basis.

This invention also discloses means for (i) rotating a small animal either a fixed or variable amount around its anterior-posterior axis, allowing the animal to be tilted, laid on either side, or laid on its back during a procedure, to facilitate certain types of stereotaxic procedures; and, (ii) using a rotary encoder to precisely measure the angle of rotation, so that any measured travel by an instrument tip can be adjusted, by mathematical calculations carried out by the software, to indicate orthogonal distances from the animal's bregma location or other fixed point, wherein the orthogonal distances are calculated relative to the animal.

Those enhancements are described below, in more detail.

Interactions with Brain Atlases

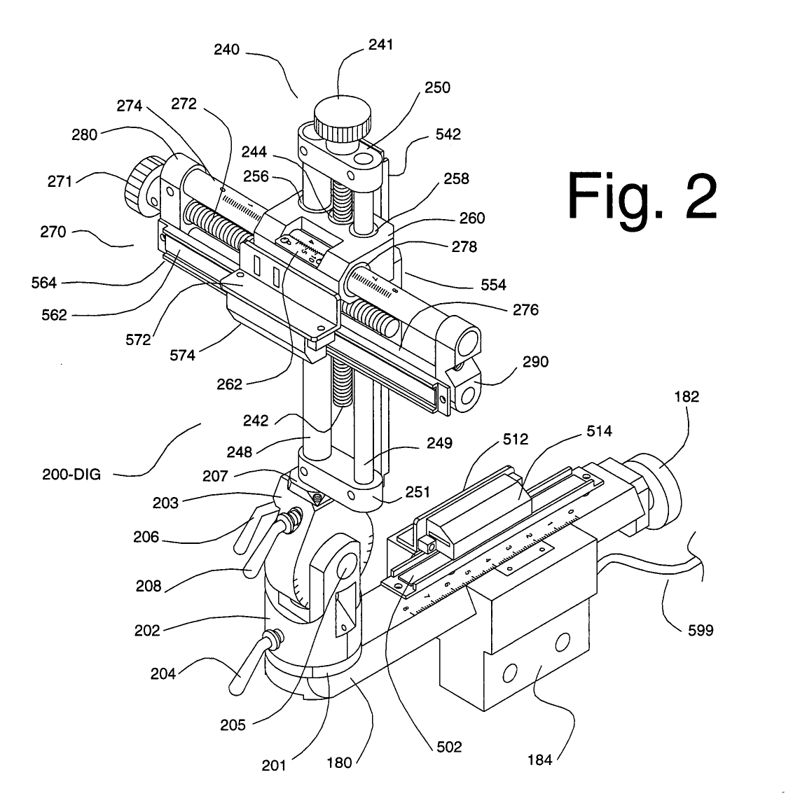

This invention discloses means for enabling a digital stereotaxic manipulator (such as manipulator 200-DIG, as shown in FIGS. 2, 3, and 8) to interact with a computer monitor that can provide high-resolution displays of digital files containing cross-sectional brain maps, from brain atlases that already have been prepared for most species of animals used in stereotaxic procedures.

In one preferred embodiment, this can be accomplished by using a data cable (such as cable 4402 in FIG. 8) to transmit data from outlet port 3916 of processor 3900 (shown in FIG. 4, and represented by interface box 4400 in FIG. 8) to a data port on a computer, such as desktop system 4000, shown in FIG. 8, which comprises a “central processing unit” (CPU) 4100, a keyboard 4200, and a monitor (or display) 4300 having monitor screen 4302.

In an alternate embodiment, it is possible to emplace, in relatively small dedicated processors, integrated circuit chips or cards that can process images, and then use a monitor cable to connect a high-resolution monitor directly to an image-handling processor. This approach could be useful, for example, to allow a large monitor screen to be positioned directly in front of the manipulator while the manipulator is being used, to create fewer visual disruptions if the operator must look closely (and alternatingly) at both the manipulator and the monitor. Accordingly, in such an embodiment, output port 3918, shown on the side of processor 3900 in FIG. 4, can represent a “Super-VGA” or comparable monitor port, to which a monitor can be connected directly, via a cable. If this approach is used, data port 3916 (which can be a USB, USB-2, Firewire, or other port) can be used to load brain map image files (which usually will be in formats such as “portable document format” (pdf) files, graphical interface (gif) files, “tagged image format” (tif) files, “bitmap” (bmp) files, etc.) into the processor, for display on the monitor. Alternately, either of ports 3916 or 3918 can be used to send instrument positioning data from the manipulator, to a separate computer, for processing and display.

As used herein, the term “high-resolution image” is defined to include any image that fills the majority of a computer monitor screen (also referred to as a display screen, or simply a monitor, display, or screen) having a diagonal size of at least about 12 inches (about 30 cm). That screen size is generally the smallest screen size conventionally used on most modern computers.

Laptop or notebook computers can be used to carry out the invention disclosed herein, if desired, especially if their software is provided with image-enlarging (“zoom”) capability to will allow an operator to enlarge an area of interest to make it more easily visible. However, most laboratories where this type of research is done are likely to have at least one computer with a monitor screen that is at least 15 inches (about 38 cm) diagonally, and many such labs have one or more monitors that are 20 inches (50 cm) or more, diagonally. Most full-color monitors have resolutions that are less than about 0.30 mm dot pitch, and many have dot pitches in the range of about 0.20 to about 0.28 mm (smaller dot pitches indicate that the pixels that create an image are packed together more tightly, and therefore provide better resolution). Some monochrome monitors have even finer resolution, and can display grayscale images with unmagnified resolutions that approach the appearance of high-quality printed pages, and can display magnified resolutions that surpass printing.

In most cases, manipulator interface 4400 as shown in FIG. 8 preferably should be a dedicated processor, such as a touch-screen device 3900 as shown in FIG. 4, which can display the orthogonal coordinates (expressed as numbers) of an instrument tip during a procedure. By displaying the coordinates of the instrument tip on this type of device, any requirement that these numbers must also be displayed on the computer monitor can be eliminated, thereby allowing the brain map being displayed on the monitor to fill all or nearly all of the monitor screen, providing the best possible resolution without cluttering or potentially confusing the map with additional data displays. In addition, especially during the early stages of development and use of the software that will allow a stereotaxic manipulator to interact with a computer showing brain maps on a monitor screen, providing an interface device that has already been extensively debugged can provide an additional safeguard, in case any anomalies appear to arise on the monitor screen that do not seem to be consistent with information the researcher believes to be valid, based on what he or she is doing and what is indicated on the dedicated processor.

However, the software that will be written to run this system preferably should be provided with options that will allow all relevant information to be displayed directly on the computer monitor, thereby allowing a researcher to simply disregard the processor display, if it is more convenient for the user to do so in some particular situation. As an example of how this can be accomplished, most graphics manipulation software allows a user to open and display any of several “toolbox” windows, within the main window that contains a picture. By using a “drag and drop” command, the user can move any such “toolbox” window to any desired area within the main “window”, and if the toolbox is in the way, the user can either close it or “minimize” it, to get it completely out of the way. Accordingly, this approach offers options that may be useful in various situations, and that are preferable in any software that will allow a computer to interact with a stereotaxic manipulator in ways that can be controlled by an operator, depending on the availability and arrangement of equipment in any particular laboratory.

Accordingly, interface device 4400 can have any of several configurations, including, as examples: (i) a dedicated processor with a relatively small touch-screen display; (ii) a free-standing box with no display screen; (iii) an interface box that is mounted on the manipulator; or (iv) an electronic card that can be inserted into a “card slot” on the mainboard of a computer. If a dedicated processor or free-standing box is used, it can be coupled to manipulator 200-DIG via cable 599, and to the computer via cable 4402, wherein each cable should be capable of handling data signals as well as providing low-power voltage to the manipulator, to drive its reader heads and rotary encoder devices. Alternately, wireless data transfer systems can be used, which preferably should use radiofrequency rather than infrared signals, to avoid any need for line-of-sight data transfer between separate units. If a wireless system is used, alternate means (such as a rechargeable battery) will be required, to provide voltage to drive the electronic devices on the manipulator.

Alternately, it is possible to completely eliminate any separate interface device, and use an existing USB port on a computer to interact with a manipulator, via a cable. USB ports and cables have become a widely used and preferred means for handling data from multiple peripherals. Many modern desktops provide four USB ports built into the mainboard, to accommodate a keyboard, pointer, and printer, with an additional port for any other device the user wants to add, and inexpensive USB hubs or routers are available, so that a single USB port on a computer can be converted into multiple ports. Among other advantages, USB interfaces can provide voltage to peripheral devices such as scanners, pointers, keyboards, etc., to drive those devices without requiring additional power cords, and USB interfaces are designed so that over 100 different peripheral devices can be run through a single computer (although few users have more than 4 or 5 USB devices coupled to a computer at one time).

Typical Procedures For Use

The following narrative is offered to explain and illustrate an example of one preferred embodiment of how this type of interaction between a stereotaxic manipulator and a computer monitor that displays a brain map can take place. This example is based on a fairly common type of procedure in neurological research, in which a researcher will insert a thin but stiff wire-like probe (generally referred to as an electrode) into a particular targeted region of a rat brain, so that the electrode can monitor nerve impulse activity within that particular brain region, during a subsequent experiment that involves a response, by the conscious animal, to some type of outside stimulus. The probe wire is regarded as part of an instrument 300 (depicted in FIG. 1), which will be temporarily secured to V-block 290 at the end of horizontal arm 270 of a digital stereotaxic manipulator 200-DIG, as shown in FIG. 2.

Prior to the start of the procedure, the researcher who will perform the procedure will plan an approach path that the instrument tip should take, through brain tissue, to minimize any damage to nerve bundles or other important structures in the animal's brain. This type of advance planning is a well-known part of such research, and is used by any skilled researcher to prepare for any neurological research that will invade an animal's brain, to minimize the damage caused by the intrusion of an instrument through brain tissue, and to maximize and protect the validity of the data gathered from the animals that are being tested.

For purposes of discussion and illustration, this example assumes that the instrument tip will need to reach a brain region that sits at a relatively low (deep) location, directly on the center sagittal plane (i.e., the vertical center plane, between the right and left hemispheres of the brain). Numerous brain regions that are of interest in research (including, for example, the hippocampus, medial thalamus, suprachiasmatic nucleus, raphe nucleus, and various others) straddle the sagittal plane, so they can serve both hemispheres equally rather than being part of one or the other. Therefore, research involving brain structures that sit directly on the center sagittal plane is common.

However, the instrument tip cannot be allowed to puncture a large blood vein called the superior sagittal sinus, positioned on top of the brain in the “centerline” crease that separates the upper surfaces of the left and right hemispheres. Therefore, an angled approach that uses a left-side or right-side entry point must be chosen and used.

Currently, nearly all invasive brain research done today uses a “flat coronal plane” approach. This means that the entire approach and retreat pathway, for an instrument tip, is constrained to a single “coronal” plane (i.e., a vertical plane that passes from an animal's right side to its left side). This procedure is standard, because it is usually the easiest way to minimize the amount of brain tissue that must be punctured by an instrument tip, as the instrument tip approaches a targeted brain region.

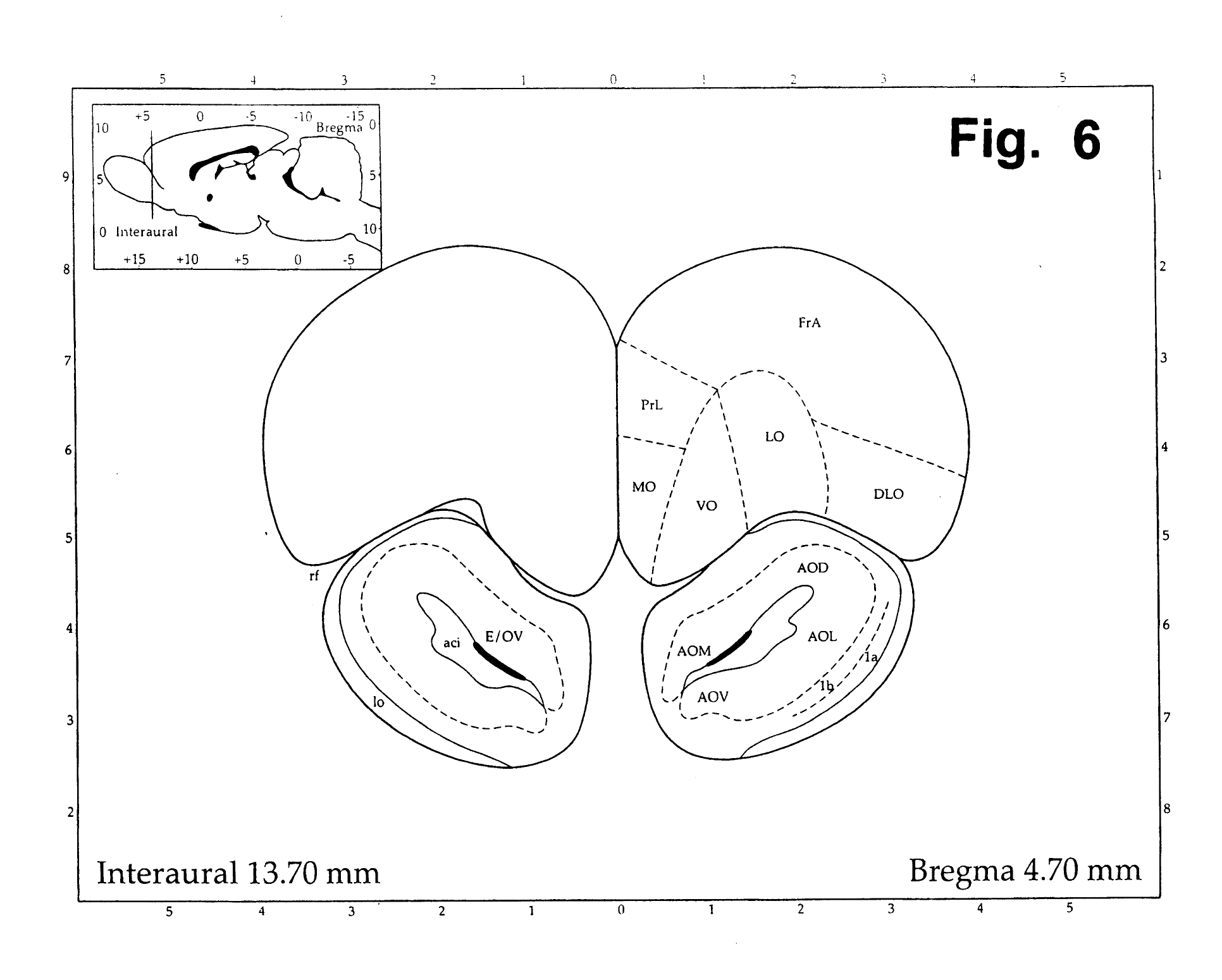

If a “flat coronal plane” approach is taken, a single cross-sectional drawing or brain map (such as shown in FIG. 6, for a mid-brain region) can be displayed throughout the entire procedure, on the computer monitor. This is depicted by brain map 4310, displayed on computer monitor screen 4302, in FIG. 8. Use of a single cross-sectional brain map, throughout an entire procedure, is possible during a “flat coronal plane” procedure, because each brain map corresponds to a single “flat coronal plane” approach. The researcher will simply choose one particular sectional drawing, from the assortment of drawings provided by the CD-ROM version of the brain atlas for the species being used, which corresponds to the location of the particular coronal plane he or she will be using, during that particular procedure.

After the selected cross-sectional brain map has been loaded onto the monitor screen 4302, as shown in FIG. 8, the rat or other animal (which is not shown in FIG. 8) can be secured in the stereotaxic holder, using ear bars 110 and 112 and snout clamp 121, as shown in FIG. 1. The scalp tissue covering the upper surface of the skull is cut and retracted, exposing the bregma (i.e., the point where the skull plate sutures cross and intersect, on top of the skull) The horizontal arm 270 of digital manipulator 200-DIG is moved into place, and any steps are taken (if necessary) to ensure that all arms and rotary encoders are set at true vertical and true horizontal positions.

Using control knobs 182, 241, and 271 (shown in FIG. 2) to control movement of slide 180, vertical arm 240, and horizontal arm 270, the instrument tip is then maneuvered until it touches the bregma point on the top of the animal's skull. A “zero-ing”command on the processor or computer is activated, and the orthogonal locations of the instrument tip, at that moment, are recorded within the computer.

It should be mentioned at this point that an optional enhancement is described below, which can allow a video image to be processed by a computer, in a way that will calculate the exact bregma location on a skull, based on the intersection between two calculated “best fit” curves, which will be determined by computer processing of a photographic image of two jagged lines. This approach can improve the accuracy of bregma point determinations, compared to visual “best guess” estimates.

The bregma coordinates in all three axes will then be used as the “zero” (or starting, baseline, reference, etc.) values, throughout the procedure. All instrument tip locations during the procedure will be measured and displayed, relative to the bregma zero-point.

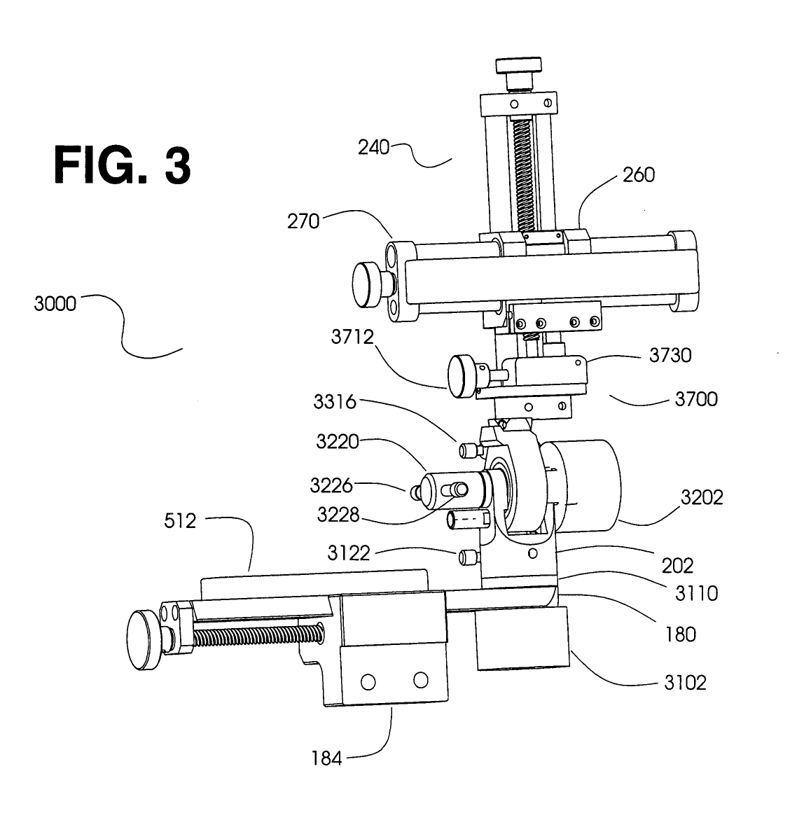

The vertical arm 240 is then tilted away from true vertical, to establish a vertically-angled approach path that was previously selected and determined by the researcher. That procedure involves loosening locking screw 3220, shown in FIG. 3, manually tilting the vertical arm assembly 240 until the data emitted by rotary encoder 3202 indicates that the desired angle has been reached, and then tightening the locking screw 3220. The angle of the vertical tilt will be recorded and stored by the processor or computer, and the sine and cosine values corresponding to that tilt angle will be used, by the processor or computer, to calculate angle-adjusted orthogonal coordinates for the instrument tip, as the procedure is carried out.

The angle-adjusted orthogonal location data for the instrument tip, which will be continuously re-calculated by the processor or computer as the instrument tip is moved by operation of the manipulator, will be superimposed on the brain map 4310 that is displayed on computer monitor 4300. At any moment during a procedure, the current location of the instrument tip can be represented, on the brain map 4310, by any clearly visible representation, such as a bright and/or blinking pinpoint of light 4320, as indicated on FIG. 8; alternately, a heavy black arrowhead or any other suitable and movable cursor, icon, or image can be used, provided that it can be easily seen by researchers who may be several meters away from the screen. For purposes of discussion, visible representation 4320 is referred to herein as instrument cursor 4320.

Since the easily-visible and movable instrument cursor 4320 will be shown by superimposing it on top of an accurate cross-sectional map 4310 of the animal brain showing the coronal plane at that position in the brain, the apparent location and movement of the instrument cursor, at any moment, can be easily and readily observed and interpreted, relative to nearby brain structures that are shown on brain map 4310. Based on the way the brain atlas was prepared, and in view of how the manipulator system, digital readers, and processing software function in an interrelated manner, the visual depiction on computer monitor screen 4302 will provide an accurate and useful depiction of where the actual instrument tip is, at any given moment, inside the brain of the animal, during an invasive procedure.

If desired, a heavy colored or black “track line” 4322 can also be displayed on monitor screen 4302. Track line 4322 can visually display a cumulative record of all locations where the instrument cursor has already passed. In most cases, this line will be a straight segment, since most procedures that involve insertion of a wire-like electrode along a straight-line pathway, since a straight-line pathway can minimize penetration of (and damage to) surrounding brain tissue. A typical straight-line approach uses manipulator slide 180 and horizontal arm 270 to position a small drill bit over a location on the rat skull where an entry hole will be drilled, and vertical control knob 241 is then used to drive a rotating drill bit through the skull. The vertical arm is then retracted, and the drill instrument is replaced by the electrode instrument. Without changing the locations of the slide 180 or the horizontal arm 270, the vertical control knob 241 and the vertical arm 240 are then used to drive the electrode through the exposed brain tissue, until the targeted location (depth) is reached, in the same straight-line pathway that was already established by the combined settings of the vertical arm tilt, the slide, and the horizontal arm. Therefore, as mentioned above, “track line” 4322 usually will be a straight line, driven entirely by motion of the vertical arm only, along a titled pathway that will be established and maintained by other components that usually will not be altered during a procedure.

It should be noted that the straightness of the track line, in this type of procedure, can also be used as a convenient way to visually confirm that all of the reader head and rotary encoder measurements, trigonometric calculations, and angle compensations are performing and interacting properly and accurately.

Potential Enhancements

If desired, the software, in conjunction with a brain map that is being displayed on a computer monitor, can be used to provide enhanced ways to help researchers use the system, while also avoiding potential dangers and problems.

For example, nerve bundles, major blood vessels, or other structures that are highly important in an animal's nervous system can be represented, on a brain map shown on a computer monitor, by vivid, blinking, or other coded colors or by similar means, rather than merely in black-and-white line drawings. This can help make sure a researcher sees and avoids the highly important structures, during a procedure. If desired, the software can cause any particular structures that have been designated with warning colors to begin blinking, and/or trigger an audible or other alarm, if an instrument tip begins approaching an important structure too closely, or if an instrument tip is heading toward a structure that must be avoided.

As a second potential enhancement, the software that comprises part of the system disclosed herein can be programmed to present “remaining gap” data which will clearly indicate how much farther an instrument tip still needs to go, to reach a targeted location. This type of information, presented in a “countdown” manner, can be highly useful and convenient to help ensure that the tip is slowed down, and then stopped, as it approaches and then reaches the exact target location.

As a third potential enhancement, the software in this system herein can be provided with an enlarge (or magnify, “zoom in”, etc.) command, that can allow any desired degree of magnification of the brain map that is being shown on the computer display. If desired, any such command can be programmed to automatically frame an area that will place the instrument tip, at that moment, at or near the center point of the enlarged display.

Although manipulators with rotary encoders are preferred for use with brain atlases as disclosed herein, digital manipulators that do not have controlled and precise angling or tilting capabilities can also be used with brain atlases, if desired. Such use, with brain atlases providing real-time visual support on a computer display, can in many cases improve the ability of researchers (including researchers who are still learning how to work with stereotaxic manipulators, either in general or with a new and unfamiliar species of animal) to carry out procedures efficiently, and with the best possible results.

Anterior-Posterior Angled Approaches

As mentioned above, nearly all invasive brain research done today uses a “flat coronal plane” approach, to minimize the amount of brain tissue that must be punctured and damaged as an instrument tip approaches a targeted brain region. This means the entire approach and retreat pathway, for an instrument tip, is constrained to a single coronal (vertical right-left) plane.

However, in some types of neurological research, it would be highly advantageous to be able to plot safer and better routes to certain brain structures, that would not be limited to a “flat coronal plane” approach.

For example, as pointed out in the Background section and as illustrated by the small cross-section drawings in the upper left corners of FIGS. 5-7, a relatively large upper cleft on the top of the brain separates the cerebral neocortex (which covers the anterior and midbrain upper layers) from the cerebellar cortex (which covers the hindbrain). If a needle, electrode, or other instrument tip could enter the brain in an angled pathway (presumably in a posterior-to-anterior direction, as the instrument tip is lowered), it could avoid both the cerebral neocortex, and the cerebellar cortex. This type of angled approach path could be used, for example, to reach the superior colliculus (which relates to vision processing), without having to puncture and penetrate the cerebral neocortex, which is also involved in vision processing.

This type of approach has not been feasible and practical, under the prior art. Under the prior art, it has not been practical or even possible to precisely control the exact angle of an instrument tip, as it approaches a certain targeted structure, using an angled pathway that violates the basic design principles of stereotaxic manipulators, which in the past have only allowed orthogonal movement and control. Since angular pathways could not be precisely established or controlled, there was no use in even trying to plot or use angular pathways that might minimize damage to the brain, but only if it could penetrate successive coronal planes of a brain in a planned, selected, and controlled manner.

However, various new possibilities and options were created by the development of controlled-angle manipulators as disclosed in application 10/636,899, and still more options can be provided, by correlating anterior/posterior angled approaches to a series of coronal plane brain maps that can be displayed, in succession, on a computer display screen.

For example, during an A/P-angled approach that passes through the large upper cleft to minimize brain tissue penetration and damage, each time the processor, based on data received from the reader heads of the manipulator, determines that an instrument tip has reached a new coronal plane that corresponds to the next available cross-sectional brain map in a series of drawings in a brain atlas, the software can be programmed to (i) automatically display an updated and selected brain map, on the monitor screen, and (ii) superimpose and display the instrument tip cursor, on the updated brain map that is being displayed.

Accordingly, these and other useful options have now been made available, for correlating the location of an instrument tip, during a stereotaxic procedure, with brain maps and atlases that already have been prepared for numerous species of animals.

Use of Contract Programmers to Write Source Code

The Inventors herein did not write the software that is used to run the myNeuroLab “Angle One” system. They have not seen the source code in that software, and indeed, they are prohibited from seeing or requesting to see that source code, under the terms of the contract that was used to obtain that software. Instead, the software that is currently being shipped to customers, as part of the dedicated PLC devices that are being sold in the “Angle One” system, was written by a contract programming company, which employs full-time professional software writers who specialize in writing software code for “programmable logic controller” (PLC) devices.

The Inventors herein gave written specifications and drawings to the software writers who specialize in writing PLC software, describing what was required and desired in the software, and providing detailed information and specifications on the design, layout, operation, and electronic components of the digitized manipulators and rotary encoders that had been selected for the system. The Inventors also provided a complete working copy of a manipulator system, having three linear reader heads and two rotary encoders, which contained the same makes and models of the reader heads and rotary encoders that had been selected by the Inventors for the systems that would be sold publicly. In addition, the Inventors provided written lists of tasks the software needed to be able to perform, written descriptions of each designated task, and written lists and descriptions of the short phrases that were desired for the operating menus and “virtual buttons” that were desired for the touch-screen display panels on the controller.

After receiving those materials and information, the software writers working for the contractor company responded by writing and debugging PLC code that carried out the instructions of the Inventors. The writing and debugging of that code required diligence and a skilled understanding of PLC programming code; however, to the best of the Inventors knowledge and belief, it did not require any undue creativity or experimentation, after the hardware and the necessary tasks for the software had been explained to the software writers, by the Inventors.

The PLC code that was purchased from the contract company is loaded (using a special machine, which is also sold by the contract company) into a proprietary type of electronically programmable memory (EPROM) device that prevents unauthorized tampering. A memory device containing the software is installed into the dedicated PLC unit sold with an “Angle One” system.

When a particular set of software code needs to be written only once, and when the software code needs to be written in a specialized programming language for devices that have markets limited to a few thousand copies (rather than in a software language used in tens or hundreds of millions of computers), the purchase of programming services from a contractor company that employs programmers with specialized skills is standard practice. It is much easier and more reliable for code-writing contractor companies to hire and retain good code writers, if a contractor can bring in numerous projects from numerous clients. As part of the contracts used to purchase such software, the contractor company typically refuses to reveal the source code for any programs written by its programmers. That was indeed one of the terms that was insisted upon, by the company that wrote the software code for the Inventors herein.

Having observed how that system and that business approach resulted in a completely satisfactory product that works quite effectively, and exactly as intended, the Inventors herein recognized how that same general approach (using software specialists who work for a contractor company that specializes in writing software programs) can be utilized again, to create an even more useful system that can merge digital stereotaxic manipulators, with brain atlas information and drawings that already have been compiled in computer-readable files.

Accordingly, to complete the reduction of this invention to practice, the Inventors herein will fully disclose the brain atlas concept and information, and will provide copies of actual brain atlases on CD-ROM's (under copyright agreements with the authors and publishers of those brain atlases), along with a working copy of an “Angle One” manipulator and processor system, to a contractor company that employs programmers who specialize in writing software in a selected programming language that is suited for performing the tasks described herein. The Inventors will also provide written lists and descriptions of the specific functions they wish to have included in the software, and how they want a digital manipulator to interact with a brain map that is being displayed on a computer monitor. The programmers who work for the contractor company will need to apply diligence and a skilled knowledge of the programming language they will be using, to write and then test and debug the code. However, to the best of the Inventors knowledge and belief, the programming work will not require any undue creativity or experimentation, once the invention, the mode of action of the desired system, and the performance needs and criteria of the system, have been explained by the Inventors, to the programmers.

As an illustration of how this system can work proprly and effectively, a simple and basic yet highly useful and effective embodiment of this invention merely requires two things:

(i) displaying, on a computer monitor, a single two-dimensional brain map that has been pre-selected by a researcher, that is already available on a CD-ROM that is sold with a published brain atlas, and that is positioned accurately within a two-dimensional coordinate grid that has X and Y axes that are measured in millimeters, and,

(ii) providing a single blinking cursor that can move across the two-dimensional coordinate grid of the brain map that is being displayed on the computer monitor, based on digital location information that is being sent to the computer, either directly from a digital stereotaxic manipulator, or from an electronic programmable interface that is positioned between the manipulatorm and the computer.

The writing of software code that can superimpose a single blinking cursor on a two-dimensional coordinate grid, based on digital signals that tells the computer exactly where the cursor should be on the grid at any moment in time, would be regarded by nearly any skilled computer programmer as a simple and elementary task, which can be carried out without requiring any undue experimentation.

Conversion of Inactive Picture Files into Active Grid Files

If the brain maps (sectional drawings) that are contained in the CD-ROM version of a certain brain atlas contain nothing more than pictorial representations (such as created by scanning a line drawing), and do not also contain the type of embedded information that is necessary to allow a movable cursor to move about the picture in a precise and accurate manner by using a two-dimensional grid that “supports” the picture, steps can be taken to convert such picture files into enhanced formats that will provide a “supporting” or “interactive” grid which the software can then use. Such conversion steps are well known to those skilled in the art of computer graphics.

For example, a file containing a scanned type of picture-only map (which does not provide a “supporting grid” for a cursor) can be enhanced, by providing it with a total of only four location points, which will correspond in a suitable manner to the four corners of a rectangular coordinate grid that contains the picture-map. As an example, the maps of rat brains that are shown in FIGS. 5-7 are consistently contained within coordinate grids that are surrounded and defined by four points. The numbers along the top and bottom horizontal axes are identical, and indicate horizontal distances from the sagittal (front-to-rear) center plane. Along the vertical axes, the “ear bar zero” numbers, shown on the left side of each drawing, are not as accurate as the bregma numbers, shown on the right side of each drawing. Therefore, the “ear bar zero” numbers should be ignored, and the bregma numbers become the only relevant vertical numbers.

Accordingly, when the horizontal X axis is used as the first number in each coordinate pair, and the vertical Y axis is used as the second number, the four corners of the mid-brain grid shown in FIG. 6 can be represented as [-8,0] for the upper left corner, [8,0] for the upper right corner, [−8,−11] for the lower left corner, and [8,−11] for the lower right corner. Accordingly, if a pictorial file is “anchored” or “pinned” to a two-dimensional computerized grid having those four corners, in a manner which ensures that each corner of the picture is anchored or pinned to the corresponding coordinates of the grid, the cursor can move about on the grid during a stereotaxic procedure, using the grid for “support”, and the cursor will be superimposed in the correct and accurate locations on the pictorial map, which uses the same grid for support.

Indeed, this type of approach requires only two location points to completely define a grid, if the grid is rectangular and is required to be “orthogonal” (i.e., with horizontal and vertical sides, rather than angled or slanting sides). A complete rectangular grid can be defined completely by a combination of either (i) the upper left and lower right corners, or (ii) the upper right and lower left corners.

In at least some programming languages, various types of tools and shortcuts are available for doing those types of conversions on an automated or semi-automated basis. This can facilitate the conversion process to point where it is simple and easy, and can be done in a manner that is analogous to: (i) inserting a picture or graphics file into a document created by a word processing program, such as Microsoft Word; (ii) resizing the picture until it is a certain size, using “preserve aspect ratio” and “snap to grid” options to make the resizing process faster and more accurate; and, (iii) storing the new file. Indeed, if all of the source files have the same size prior to conversion, many programs will allow “macro” or “hotkey” programs to be created and used, which will carry out the entire series of steps when activated by a combination keystroke, such as Alt-Z or. Control-Z.

Accordingly, programmers can work with any type of pdg, gif, tif, bitmap, or other pictorial files that are provided as computerized files with any particular brain atlas. Those files can be converted into (or anchored or “pinned” to) two-dimensional grids, in a manner that will support accurate depictions of cursor locations and movements, superimposed on the pictures, during a stereotaxic procedure.

Use of Video Processing to Determine Exact Location of Bregma

On the subject of graphical manipulation of picture files, it should be noted that the machinery and software disclosed herein, with one additional enhancement, can also be used to establish an exact location of the bregma point, on top of an animal skull. This enhancement can be useful, since it is sometimes difficult to determine an exact bregma point by visual examination of a skull.

The problem that is addressed and solved by this enhancement involves the following facts. The bregma is determined by the intersection of two perpendicular “fissure” or “suture” lines, where four different bone plates fused together, to form the top of a mouse, rat, or other animal skull. In order to provide the complete skull bone with greater strength, after those plates have grown together, these two fissure lines have jagged and zig-zagging shapes, in a manner analogous to interlocking fingers. While these nonlinear interlocking lines do indeed provide stronger connections an dinterfaces, the fact that they are jagged, non-linear, and irregular can make it very difficult to determine a precise bregma point. Accordingly, a researcher today must try to visually assess the appearance of the lines, and then must try to place an instrument tip at the “best guess” visually estimated location.

This type of visual approximation is usually reliable, down to a fraction of a millimeter. However, even if a researcher's “best guess” is accurate to within 0.1 mm (which is quite accurate, for a visual estimate), a discrepancy of only 0.1 mm still represents an error of 100 microns, and it must be kept in mind that each and every measurement, during an entire procedure, will be based entirely on the researcher's “best guess” estimate as to where the bregma point was. Since digital measurements accurate to a single micron can be achieved quickly, consistently, and reliably, using digital stereotaxic manipulators, better accuracy and precision for all measurements throughout an entire procedure can be provided by using a computer to determine the starting-point bregma location, to within an accuracy of only one or a few microns.

This can be done by steps such as the following:

(1) A digital video camera is temporarily but securely positioned above the skull;

(2) The lighting in the room is controlled so that the light falls on the skull from a different angle, causing the jagged fissure lines to be clearly visible, as dark shadowed lines across the top of the skull;

(3) A digital photograph is taken of the top of the exposed animal skull, by the stationary camera;

(4) The photograph is magnified and displayed on a computer monitor, in a grid-anchored photograph, in a way that causes a rectangle that is roughly 1 centimeter square, on top of the skull, to fills the entire monitor screen;

(5) The contrast and brightness controls are adjusted and manipulated by the operator, until the darkened shadowed lines at the fissures become thin and starkly black, forming a high-contrast black-and-white image;

(6) The high-contrast image is processed, to convert the successive “fingertip points” on each of the two jagged suture lines (which must be kept separate and distinct from each other, in this processing step) into a series of coordinates, on the two-dimensional grid;

(7) The collection of coordinate locations, representing the “fingertip points” on each of the two fissure lines, is then processed, by a mathematical algorithm, to determine a “best fit” curve that gives the closest “smoothed-out” curve for each jagged fissure line.

The intersection of the two “best fit” curves will then provide the best and most accurate location, for the bregma point.

All of the foregoing can be done while the video camera remains locked in the same position. After the calculated bregma point has been determined, using the steps listed above, the screen is then returned to a magnified live image, showing exactly the same photograph, with the two calculated curves superimposed on that image. The operator then operates the manipulator, until the instrument tip (which will appear on the video image, in a live image) touches the skull bone at the exact location where the two calculated curves appear, superimposed on the nonmoving bone which is being displayed on the computer monitor.

Accordingly, this method can be used to provide practical and relatively inexpensive means for establishing the bregma location at a point that has been analyzed and determined by a computer to be the bregma location, accurate to within a few microns, rather than relying on a visual assessment to give a “best estimate” that may be off by hundreds of microns.

Precise Rotation of Snout Clamp

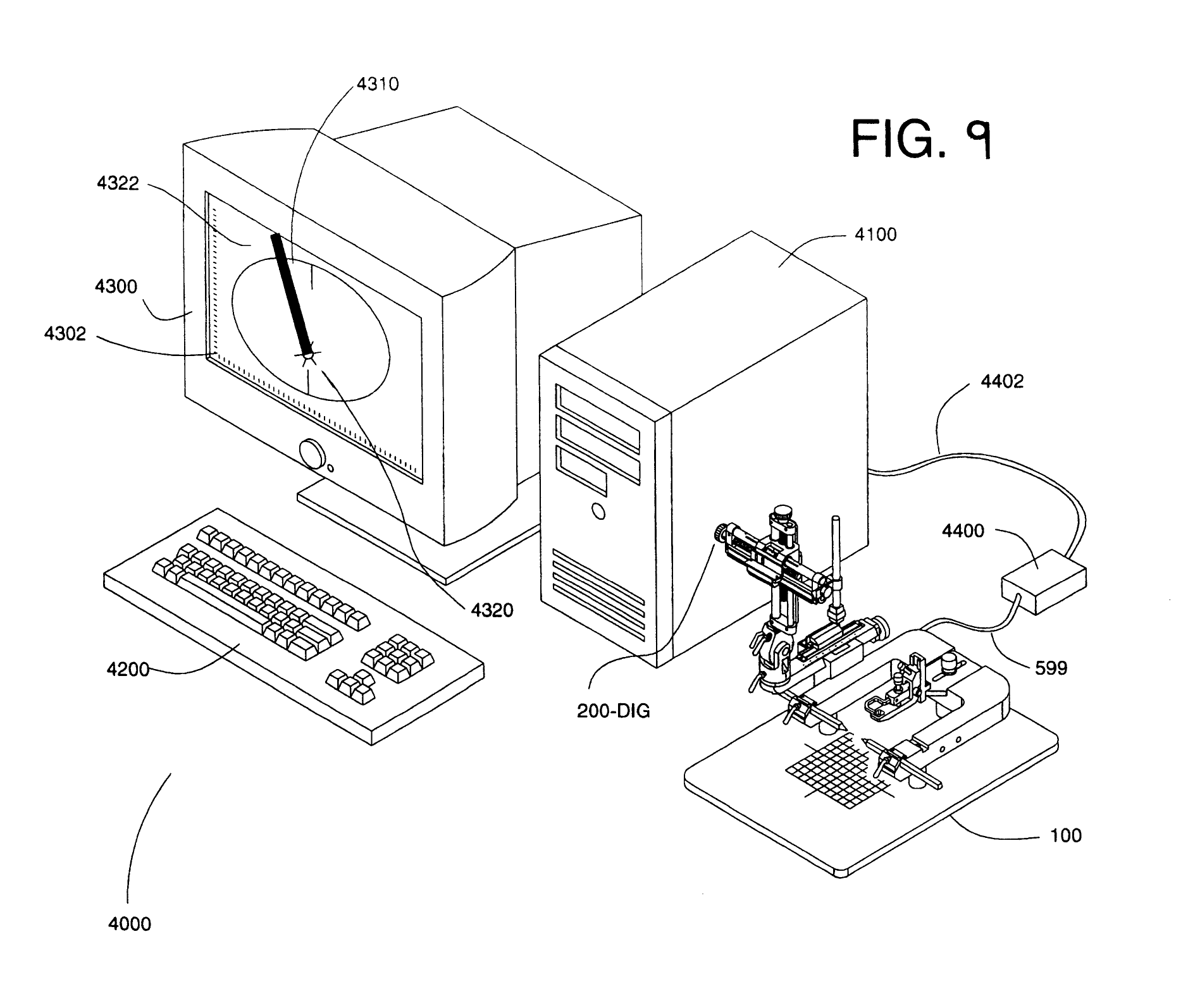

FIG. 9 depicts an enhanced animal holder 100R, having a conventional base plate 102 and U-frame 104. In this enhanced device, a snout clamp 121 (usually comprising a movable upper bar, mounted above a lower plate with a rectangular hole that accommodate the upper front teeth of a rodent) is affixed to U-frame 104 by means of a rotatable axle, which has an anterior-posterior (A/P) orientation. This axle component (which is already present, in many types of commercially available holders) allows an animal to be rotated around the A/P axis, so that either side or the belly of the animal faces upward.

In enhanced holder 100R, a rotary encoder 3800 is provided, to precisely measure the angle of rotation of the snout clamping assembly (including snout clamp 121) around the rotatable A/P axle.

This can facilitate (and in some cases enable) various types of procedures that are much more difficult if an animal is in a prone position, with its back on top, and with large parts of its brain sitting directly on top of various lower brain structures that are of great interest to researchers. As an example, as described above, when an animal is in a flat prone position, a vertical approach path along the center sagittal plane would need to puncture the major blood vein (the sagittal sinus) that travels along the top of the brain, in the crease between the tops of the two hemispheres.

Accordingly, rotary encoder 3800 can allow the tilting or rotation of the animal, along the A/P axis, to be included among the data that are being processed by a processor or computer, during a procedure. This can enable angle-adjusted orthogonal data (relative to the animal) to be generated and displayed, even when the animal is in a tilted, sideways, or upside-down position.

Thus, there has been shown and described a new and useful means for enabling stereotaxic manipulators to interact with brain maps that are being displayed on computer screens. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Morin, L., and Wood, R., Stereotaxic Atlas of the Golden Hamster Brain (with CD-ROM; Academic Press, 2001)
Paxinos, G., and Watson, C., The Rat Brain in Stereotaxic Coordinates (fourth edition with CD-ROM; Academic Press, 1998)
Paxinos, G., and Franklin, K., The Mouse Brain in Stereotaxic Coordinates (with CD-ROM; Academic Press, 1998)
Paxinos, G., et al, The Rhesus Monkey Brain in Stereotaxic Coordinates (with CD-ROM; Academic Press, 1999)

1. A computerized stereotaxic manipulator system suited for neurologic research on non-human animals, comprising:
a. a stereotaxic manipulator having electronic components mounted thereon which can emit electronic signals that can be processed to indicate positioning and motion of an instrument tip, during a neurological procedure while said positioning and motion are controlled by components of the stereotaxic manipulator;
b. a computerized processing and display system, comprising signal-processing circuitry, software, and a monitor, that are capable of interacting together during a neurological procedure, to:
(i) display, in high-resolution form on the monitor, an accurate cross-sectional image of an animal brain; and,
(ii) display on the monitor, in a manner that is correlated with the cross-sectional image of the animal brain, a visual symbol which indicates the positioning and motion of the instrument tip, relative to the animal brain, during a neurological procedure.

2. The computerized stereotaxic manipulator system of claim 1, wherein the neurological procedure is constrained to a single flat coronal plane, and wherein the cross- sectional image of the animal brain being displayed on the monitor during the neurological procedure corresponds to the flat coronal plane where the instrument tip is penetrating brain tissue in the animal brain.

3. The computerized stereotaxic manipulator system of claim 1, wherein the instrument tip traverses a series of coronal planes during the neurological procedure, reflecting motion of the instrument tip along an anterior-posterior axis during the neurological procedure, and wherein the cross-sectional image of the animal brain that is being displayed on the monitor during the neurological procedure is updated periodically in a manner that reflects travel of the instrument tip along the anterior-posterior axis during the neurological procedure.

4. The computerized stereotaxic manipulator system of claim 1, wherein the software enables display, during the neurological procedure, of numerical distances, along all three orthogonal axes, which indicate current orthogonal distances of the instrument tip, during the neurological procedure, from a predetermined target location inside the animal brain.

5. The computerized stereotaxic manipulator system of claim 1, wherein the software is programmed to enable specific designated regions within a cross-sectional image of an animal brain to be displayed on the monitor using visual means that visually distinguish such specific designated regions from nondesignated regions of the animal brain.

6. The computerized stereotaxic manipulator system of claim 1, wherein the software is programmed to activate a warning signal if an instrument tip approaches, within a predetermined proximity, a specific designated region of an animal brain.

7. The computerized stereotaxic manipulator system of claim 1, wherein the software is programmed to activate a warning signal if an instrument tip is travelling on a linear pathway that will approach, within a predetermined proximity, a specific designated region of an animal brain.

8. A method of performing neurologic research on non-human animals, comprising:
a. using a stereotaxic manipulator to manipulate an instrument tip during a neurologic procedure, wherein said stereotaxic manipulator has electronic components mounted thereon which can emit electronic signals that can be processed to indicate positioning and motion of an instrument;
b. using a computerized processing and display system, comprising signal-processing circuitry, software, and a monitor, to simultaneously display on the monitor: (i) a cross-sectional image of an animal brain, and (ii) a visual symbol which indicates the positioning and motion of the instrument tip, within the animal brain, during the neurologic procedure.

9. The method of claim 8, wherein the neurological procedure is constrained to a single flat coronal plane, and wherein the cross-sectional image of the animal brain being displayed on the monitor during the neurological procedure corresponds to the flat coronal plane where the instrument tip is penetrating brain tissue in the animal brain.

10. The method of claim 8, wherein the instrument tip traverses a series of coronal planes during the neurological procedure, reflecting motion of the instrument tip along an anterior-posterior axis during the neurological procedure, and wherein the cross-sectional image of the animal brain that is being displayed on the monitor during the neurological procedure is updated periodically in a manner that reflects travel of the instrument tip along the anterior-posterior axis during the neurological procedure.

11. The method of claim 8, wherein the software enables display, during the neurological procedure, of numerical distances, along all three orthogonal axes, which indicate current orthogonal distances of the instrument tip, during the neurological procedure, from a predetermined target location inside the animal brain.

12. The method of claim 8, wherein the software is programmed to enable specific designated regions within a cross-sectional image of an animal brain to be displayed on the monitor using visual means that visually distinguish such specific designated regions from nondesignated regions of the animal brain.

13. The method of claim 8, wherein the software is programmed to activate a warning signal if an instrument tip approaches, within a predetermined proximity, a specific designated region of an animal brain.

14. The method of claim 8, wherein the software is programmed to activate a warning signal if an instrument tip is travelling on a linear pathway that will approach, within a predetermined proximity, a specific designated region of an animal brain.
